(19) United States
(12) Patent Application Publication
(10) Pub. No.: US 2019/0353669 A1
(43) Pub. Date: Nov. 21, 2019

(54) Detection of Brain Injury or Neurological Disease using Tau Protein

(71) Applicant: Amprion, Inc., San Francisco, CA (US)

(72) Inventors: Russell M. Lebovitz, Oakland, CA (US); Benedikt K. Vollrath, San Diego, CA (US); Luis Concha, San Diego, CA (US); Claudio Soto-Jara, Friendswood, TX (US)

(21) Appl. No.: 16/414,749

(22) Filed: May 16, 2019

Related U.S. Application Data
(60) Provisional application No. 62/672,343, filed on May 16, 2018.

(51) Int. Cl. G01N 33/68

(52) U.S. Cl. CPC G01N 33/6896; G01N 33/6818; G01N 33/6851; G01N 2333/4709; G01N 2800/2835; G01N 2800/2871; G01N 2800/2821

(57) ABSTRACT

Methods and kits for evaluating a subject for a brain injury are described. The method may include providing at least one biological sample from the subject having or suspected of having the brain injury. The method may include conducting one or more amplification reactions, including contacting a portion of the biological sample with a monomeric, folded tau protein to form an incubation mixture. Each amplification reaction may include determining a presence or amount of the misfolded tau protein in the biological sample according to the amplified portion of the misfolded tau protein. Methods for evaluating the risk of neurodegenerative disease or disorder in a subject having suffered from brain injury are also described.

### CROSS-REFERENCE TO RELATED APPLICATIONS

This application claims priority from U.S. Provisional Patent Application No. 62/672,343, filed on May 16, 2018 the entire contents of which are incorporated herein by reference.

### STATEMENT AS TO FEDERALLY SPONSORED RESEARCH

This invention was made with government support under AG058333 awarded by the National Institutes of Health. The government has certain rights in the invention.

### BACKGROUND

Many neurodegenerative conditions are associated with brain injury. For example, neurodegenerative conditions associated with acute brain injury may arise from acute instances of mechanical damage from contact sports, accidents, and violence, as in traumatic brain injury (TBI). Acute brain injury may be associated with infection or inflammation, for example, as in encephalitis and meningitis. Chronic brain injury may be associated with disease conditions that involve steady state mechanical damage, for example, non-communicating hydrocephalus and normal pressure hydrocephalus. Chronic brain injury may also arise from repeated instances of mechanical damage, such as chronic traumatic encephalopathy (CTE), chronic traumatic encephalomyopathy (CTEM), dementia pugilistica, and the like, which may be associated with symptomatic concussions, and the like. Some current reports indicate that significant brain injury may arise from asymptomatic sub-concussive incidents.

The neurodegeneration associated with such various forms of brain injury may include problems with functions controlled or mediated by the brain, such as behavior, mood, perception, memory, pain, cognition, voluntary and involuntary bodily control, and the like. Moreover, such neurodegeneration may be progressive. However, definitive diagnosis of such neurodegeneration has to date only been feasible by analysis of brain tissue post mortem, where, for example, CTE has been observed to be associated with deposits of misfolded tau protein. There is at present no way of detecting or differentiating neurodegenerative conditions arising from brain injury while the patient is alive, let alone monitoring and managing brain injury that may be asymptomatic, or such neurodegeneration at a pre-clinical stage—for example, CTE associated with contact sports may not manifest as clinical symptoms until years or decades later. Moreover, there is currently no way of differentiating neurodegenerative conditions associated with brain injury involving mechanical damage such as impact or pressure, from chronic neurodegenerative diseases such as Alzheimer's disease and Parkinson's disease that, by contrast with brain injury, are primarily associated with chronic biochemical damage.

It is estimated that 1.5 million individuals sustain a traumatic brain injury (TBI) annually. Many of these individuals do not have visible physical signs while suffering effects such as behavioral impairments or employment disabilities and thus the brain injury is often unnoticed or misdiagnosed. Fortunately 80-90% of patients sustaining a mild TBI fully recover from their injury. However, for the remaining 10-20% of patients who continue to have persistent symptoms, accurate diagnosis may lead to improved clinical outcomes.

Imaging such as a CT or MRI may be obtained if a patent has suffered a brain injury under certain criteria such as a loss of consciousness. These scans however, are mainly aimed at identifying macroscopic lesions and are limited in their capacity to assess microscopic which matter injuries. Neuropsychological evaluations that examine attention, speech language, memory, executive function and though processing can be time consuming to conduct. Furthermore, current diagnostic tests are neither sensitive nor specific enough to identify individuals with a mild traumatic brain injury. Accurate and timely diagnosis would be of immense assistance in distinguishing individuals who have suffered a traumatic brain injury to provide appropriate and immediate medical intervention.

Brain injury and associated conditions such as neurodegeneration can be difficult to detect. Accordingly, there remains a need for methods of detecting brain injury and associated pathologies such as neurodegeneration.

### SUMMARY

In one aspect, the present invention provides a method for evaluating a subject for a brain injury. The method includes the steps of providing at least one biological sample from the subject; conducting one or more amplification reactions, each amplification reaction comprising: contacting a portion of the biological sample with a monomeric, tau protein to form an incubation mixture; subjecting the incubation mixture to amplification conditions that are effective, in the presence of the misfolded tau protein, to form an amplified portion of the misfolded tau protein from the monomeric tau protein; detecting the amplified portion of the misfolded tau protein; determining the presence or amount of the misfolded tau protein in the biological sample by detecting the presence or amount of the amplified portion of the misfolded tau protein; and characterizing the subject as having an increased risk of having brain injury if misfolded tau protein is determined to be present in the biological sample. In some embodiments, the tau protein comprises a 3R tau protein. In further embodiments, the tau protein comprises a 4R tau protein.

In some embodiments, characterizing the risk of brain injury in the subject further comprises classifying the misfolded tau protein according to one or more features comprising: an amino acid sequence, a post translational modification (PTM), an isoform, a misfolding conformation variant, an aggregation variant; and an amplification kinetics parameter. In further embodiments, the misfolded tau protein is classified using one or more of: protein sequencing; an antibody; an indicator; chemical analysis of the PTM; a spectrum; microscopy, a proteolytic resistance; a stability to denaturation; and a kinetics analysis of the one or more amplification reactions.

In some embodiments, conducting the one or more amplification reactions comprise conducting two or more amplification reactions; and characterizing the risk of brain injury in the subject by comparing, between each of the two or more amplification reactions, the presence or amount of misfolded tau protein in each biological sample. In additional embodiments, conducting the one or more amplification reactions comprises conducting two or more amplification reactions, the misfolded tau protein being distinguished between the two or more amplification reactions according to one or more features comprising: an amino acid sequence, a post translational modification (PTM), an isoform, a misfolding conformation variant, an aggregation variant; and an amplification kinetics parameter; and characterizing the risk of brain injury in the subject comprises comparing, between the two or more amplification reactions, the presence or amount of misfolded tau protein in each biological sample.

In some embodiments, the method further comprises obtaining the at least one biological sample from the subject. In further embodiments, the method comprises providing a separate biological sample for each of the one or more amplification reactions. In yet further embodiments, the biological sample comprises cerebrospinal fluid. In some embodiments, the one or more amplification reactions comprise protein misfolding cyclic amplification (PMCA) and/or quaking-induced conversion (QuIC).

In some embodiments, the subject is suspected as having brain injury as a result of having suffered from trauma to the brain selected from the group consisting of traumatic brain injury (TBI), acute infection, acute inflammation, encephalitis, meningitis, non-communicating hydrocephalus, normal pressure hydrocephalus, chronic traumatic encephalopathy (CTE), chronic traumatic encephalomyopathy (CTEM), dementia pugilistica, a symptomatic concussion, and an asymptomatic sub-concussive incident. In further embodiments, the biological sample is obtained from the subject within 24 hours from the occurrence of the brain trauma.

In some embodiments, the method includes the step of selectively concentrating the misfolded tau protein in one or more of the biological samples and the incubation mixture. In further embodiments, selectively concentrating the misfolded tau protein comprises one or more of: pre-treating the biological sample prior to forming the incubation mixture; pre-treating the incubation mixture prior to incubating the incubation mixture; and contacting one or more misfolded tau protein-specific antibodies to the misfolded tau protein to form a captured misfolded tau protein, the one or more misfolded tau protein-specific antibodies comprising one or more of: an antibody specific for an amino acid sequence of the misfolded tau protein and an antibody specific for a conformation of the misfolded tau protein.

In some embodiments, a subject identified as having an increased risk of brain injury using a method of the invention is provided with treatment for brain injury. In a further embodiment, the method comprises comparing the amount of the misfolded tau protein in the biological sample to an amount of the misfolded tau protein in a comparison biological sample, the biological sample and the comparison biological sample being taken from the subject at different times over a period of time under the treatment; and determining if the subject is responsive to the treatment according to a change in the misfolded tau protein over the period of time, or non-responsive to the treatment according to homeostasis of the misfolded tau protein over the period of time.

In another aspect, the present invention provides a method for evaluating the risk of neurodegenerative disease or disorder in a subject having suffered from brain injury. The method includes the steps of providing one or more biological samples from the subject; conducting one or more amplification reactions, each amplification reaction comprising: contacting a portion of the biological sample with a monomeric, folded tau protein to form an incubation mixture; subjecting the incubation mixture to amplification conditions that are effective, in the presence of the misfolded tau protein, to form an amplified portion of the misfolded tau protein from the monomeric, folded tau protein; detecting the amplified portion of the misfolded tau protein; determining a presence or amount of the misfolded tau protein in the biological sample according to the amplified portion of the misfolded tau protein; and characterizing the risk of neurodegenerative disease in the subject according to the presence or amount of the misfolded tau protein in the biological sample. In some embodiments, the brain injury is traumatic brain injury. In further embodiments, the traumatic brain injury is chronic traumatic encephalopathy. In additional embodiments, the subject is exhibiting the one or more clinical signs of dementia according to cognitive testing.

In some embodiments, the neurological disease or disorder is a tauopathy selected from the group consisting of Alzheimer's disease (AD), Parkinson's Disease (PD), Progressive Supranuclear Palsy (PSP), FrontoTemporal Dementia (FTD), Corticobasal degeneration (CBD), Mild cognitive impairment (MCI), Argyrophilic grain disease (AgD) Traumatic Brain Injury (TBI), Chronic Traumatic Encephalopathy (CTE), and Dementia Pugilistica (DP).

### BRIEF DESCRIPTIONS OF THE DRAWINGS

The present invention may be more readily understood by reference to the following figures, wherein:

FIG. 1 provides a graph showing the detection of Tau seeds in the cerebrospinal fluid of a patient affected by traumatic brain injury (TBI). The Tau-PMCA assay was performed on 96 well plates using 12.5 μM Tau monomer, 1.25 mM heparin, 5 mM Thioflavin T, using cyclic agitation (1 min shaking at 500 rpm followed by 29 min without shaking). Samples of 25 I from a TBI patient as well as 3 controls were added. Aggregation was followed over time by ThT fluorescence. The Graph shows the mean and standard error of three replicates.

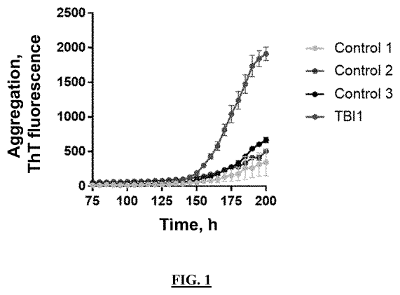

### DETAILED DESCRIPTION

Methods, compositions, and kits are provided for the detection of misfolded tau proteins in a sample, including for the evaluation of brain injury in a subject. Brain injury may be associated with misfolded tau protein, which may be formed upon acute or chronic brain injury and may accumulate thereafter. The misfolded tau protein may induce cellular dysfunction and tissue damage, either alone or in misfolded tau protein aggregates, potentially leading to the development of a neurological disease or disorder.

### Definitions

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “selectively” is used in the specification or the claims, it is intended to refer to a condition of a component wherein a user of the apparatus may activate or deactivate the feature or function of the component as is necessary or desired in use of the apparatus. To the extent that the term “operatively connected” is used in the specification or the claims, it is intended to mean that the identified components are connected in a way to perform a designated function. To the extent that the term “substantially” is used in the specification or the claims, it is intended to mean that the identified components have the relation or qualities indicated with degree of error as would be acceptable in the subject industry.

As used in the specification and the claims, the singular forms “a,” “an,” and “the” include the plural unless the singular is expressly specified. For example, reference to “a compound” may include a mixture of two or more compounds, as well as a single compound.

As used herein, the term “about” in conjunction with a number is intended to include ±10% of the number. In other words, “about 10” may mean from 9 to 11.

As used herein, the terms “optional” and “optionally” mean that the subsequently described circumstance may or may not occur, so that the description includes instances where the circumstance occurs and instances where it does not.

In addition, where features or aspects of the disclosure are described in terms of Markush groups, those skilled in the art will recognize that the disclosure is also thereby described in terms of any individual member or subgroup of members of the Markush group. As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, and the like. As a non-limiting example, each range discussed herein can be readily broken down into a lower third, middle third and upper third, and the like. As will also be understood by one skilled in the art all language such as “up to,” “at least,” “greater than,” “less than,” include the number recited and refer to ranges which can be subsequently broken down into sub-ranges as discussed above. Finally, as will be understood by one skilled in the art, a range includes each individual member. For example, a group having 1-3 cells refers to groups having 1, 2, or 3 cells. Similarly, a group having 1-5 cells refers to groups having 1, 2, 3, 4, or 5 cells, and so forth. While various aspects and embodiments have been disclosed herein, other aspects and embodiments will be apparent to those skilled in the art.

As used herein, the terms “treatment”, “treating”, and the like, refer to obtaining a desired pharmacologic or physiologic effect. The effect may be therapeutic in terms of a partial or complete cure for a disease or an adverse effect attributable to the disease. “Treatment”, as used herein, covers any treatment of a disease in a mammal, particularly in a human, and can include inhibiting the disease or condition, i.e., arresting its development; and relieving the disease, i.e., causing regression of the disease.

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

A “subject,” as used herein, can be any mammal (e.g., a rat or deer), such as a domesticated farm animal (e.g., cow, horse, pig) or pet (e.g., dog, cat). In some embodiments, the subject is a non-human primate, while in a preferred embodiment the subject is a human. The subject can also be referred to herein as the patient.

The terms “polypeptide” and “peptide” are used interchangeably herein to refer to a polymer of amino acids. These terms do not connote a specific length of a polymer of amino acids. Thus, for example, the terms oligopeptide, protein, and enzyme are included within the definition of polypeptide or peptide, whether produced using recombinant techniques, chemical or enzymatic synthesis, or naturally occurring. This term also includes polypeptides that have been modified or derivatized, such as by glycosylation, acetylation, phosphorylation, and the like.

As used herein, a “misfolded tau protein aggregate” is a protein that contains in part or in full a structural conformation of the protein that differs from the structural conformation that exists when involved in its typical, non-pathogenic normal function within a biological system. A misfolded tau protein may aggregate. A misfolded tau protein may localize in a protein aggregate. A misfolded tau protein may be a non-functional protein. A misfolded tau protein may be a pathogenic conformer of the protein. Monomeric protein compositions may be provided in native, nonpathogenic conformations without the catalytic activity for misfolding, oligomerization, and aggregation associated with seeds (a misfolded tau protein oligomer capable of catalyzing misfolding under PMCA conditions). Monomeric protein compositions may be provided in seed-free form.

As used herein, “monomeric protein” refers to single protein molecules. “Soluble, aggregated misfolded tau protein” refers to oligomers or aggregations of monomeric protein that remain in solution. Examples of soluble, misfolded tau protein may include any number of protein monomers so long as the misfolded tau protein remains soluble. For example, soluble, misfolded tau protein may include monomers or aggregates of between 2 and about 50 units of monomeric protein.

Monomeric and/or soluble, misfolded tau protein may aggregate to form insoluble aggregates, higher oligomers, and/or tau fibrils. For example, aggregation of tau protein may lead to protofibrils, fibrils, and eventually misfolded plaques or tangles that may be observed in tauopathy subjects. “Seeds” or “nuclei” refer to misfolded tau protein or short fragmented fibrils, particularly soluble, misfolded tau protein with catalytic activity for further misfolding, oligomerization, and aggregation. Such nucleation-dependent aggregation may be characterized by a slow lag phase wherein aggregate nuclei may form, which may then catalyze rapid formation of further aggregates and larger oligomers and polymers. The lag phase may be minimized or removed by addition of pre-formed nuclei or seeds. Monomeric protein compositions may be provided without the catalytic activity for misfolding and aggregation associated with misfolded seeds. Monomeric protein compositions may be provided in seed-free form.

As used herein, “soluble” species may form a solution in biological fluids under physiological conditions, whereas “insoluble” species may be present as precipitates, fibrils, deposits, tangles, or other non-dissolved forms in such biological fluids under physiological conditions. Such biological fluids may include, for example, fluids, or fluids expressed from one or more of: amniotic fluid; bile; blood; cerebrospinal fluid; cerumen; skin; exudate; feces; gastric fluid; lymph; milk; mucus, e.g. nasal secretions; mucosal membrane, e.g., nasal mucosal membrane; peritoneal fluid; plasma; pleural fluid; pus; saliva; sebum; semen; sweat; synovial fluid; tears; urine; and the like. Insoluble species may include, for example, fibrils of 4R tau, 3R tau, combinations thereof (3R tau+4R tau). A species that dissolves in a non-biological fluid but not one of the aforementioned biological fluids under physiological conditions may be considered insoluble. For example, fibrils of 4R tau, 3R tau, combinations thereof may be dissolved in a solution of, e.g., a surfactant such as sodium dodecyl sulfate (SDS) in water, but may still be insoluble in one or more of the mentioned biological fluids under physiological conditions.

In some embodiments, the sample may exclude insoluble species of the misfolded tau proteins such as 4R tau, 3R tau, combinations thereof as a precipitate, fibril, deposit, tangle, plaque, or other form that may be insoluble in one or more of the described biological fluids under physiological conditions.

For example, in some embodiments, the sample may exclude tau in fibril form. The sample may exclude misfolded tau proteins in insoluble form, e.g., the sample may exclude the misfolded tau proteins as precipitates, fibrils, deposits, tangles, plaques, or other insoluble forms, e.g., in fibril form. The methods described herein may include preparing the sample by excluding the misfolded tau protein in insoluble form, e.g., by excluding from the sample the misfolded tau protein as precipitates, fibrils, deposits, tangles, plaques, or other insoluble forms, e.g., in fibril form. The kits described herein may include instructions directing a user to prepare the sample by excluding from the sample the misfolded tau protein as precipitates, fibrils, deposits, tangles, plaques, or other insoluble forms, e.g., in fibril form. The exclusion of such insoluble forms of the described misfolded tau proteins from the sample may be substantial or complete.

As used herein, aggregates of misfolded tau protein refer to non-covalent associations of protein including soluble, misfolded tau protein. Aggregates of misfolded tau protein may be “de-aggregated”, or disrupted to break up or release soluble, misfolded tau protein. The catalytic activity of a collection of soluble, misfolded tau protein seeds may scale, at least in part with the number of such seeds in a mixture. Accordingly, disruption of aggregates of misfolded tau protein in a mixture to release misfolded tau protein seeds may lead to an increase in catalytic activity for oligomerization or aggregation of monomeric protein.

### Methods for Evaluating a Subject for Brain Injury

In one aspect, the present invention provides a method for evaluating a subject for a brain injury. The method includes the steps of providing at least one biological sample from the subject; conducting one or more amplification reactions, each amplification reaction comprising: contacting a portion of the biological sample with a monomeric, tau protein to form an incubation mixture; subjecting the incubation mixture to amplification conditions that are effective, in the presence of the misfolded tau protein, to form an amplified portion of the misfolded tau protein from the monomeric tau protein; detecting the amplified portion of the misfolded tau protein; determining the presence or amount of the misfolded tau protein in the biological sample by detecting the presence or amount of the amplified portion of the misfolded tau protein; and characterizing the subject as having an increased risk of having brain injury if misfolded tau protein is determined to be present in the biological sample.

Evaluating a subject for brain injury includes diagnosing the likelihood that the subject has brain injury and/or characterizing the nature of the brain injury. A subject has an increased risk of having a brain injury if misfolded tau protein is determined to be present in the biological sample obtained from the subject. The presence of amount of misfolded tau protein can also be used the characterize, for example, whether or not the brain injury is a primary or secondary brain injury, and whether it is the result of a transmissible spongiform encephalopathy disease.

Brain injury is the destruction or degeneration of brain cells. Brain injuries occur due to a wide range of internal and external factors. Brain injury includes both significant, undiscriminating trauma-induced damage, and selective, chemically induced neuron damage. A common category with the greatest number of injuries is traumatic brain injury (TBI) following physical trauma or head injury from an outside source, and the term acquired brain injury (ABI) is used by those skilled in the art to differentiate brain injuries occurring after birth from injury, from a genetic disorder, or from a congenital disorder. Brain injury can be categorized as primary and secondary brain injuries to identify the processes involved, and further categorized as focal and diffuse brain injury to describe the severity and localization. Primary brain injury occurs during the initial insult, and results from displacement of the physical structures of the brain. Secondary brain injury occurs gradually and may involve an array of cellular processes.

Brain injury may include, for example, traumatic brain injury (TBI), acute infection, acute inflammation, encephalitis, meningitis, non-communicating hydrocephalus, normal pressure hydrocephalus, chronic traumatic encephalopathy (CTE), chronic traumatic encephalomyopathy (CTEM), dementia pugilistica, a symptomatic concussion, and an asymptomatic sub-concussive incident.

In some embodiments, brain injury may be associated with infectious prion diseases, e.g., transmissible spongiform encephalopathy diseases (TSE). Such transmissible spongiform encephalopathies (TSE) are a group of infectious neurodegenerative diseases that affect humans and animals. For example, human TSE diseases may include: Creutzfeldt-Jakob disease and its variant (CJD, vCJD), kuru, Gerstmann-Straussler-Scheiker disease (GSS), and fatal familial insomnia (FFI). Animal TSE diseases may include sheep and goats (scrapie); cattle (bovine spongiform encephalopathy, BSE); elk, white-tailed deer, mule deer and red deer (Chronic Wasting Disease, CWD); mink (transmissible mink encephalopathy, TME); cats (feline spongiform encephalopathy, FSE); nyala and greater kudu (exotic ungulate encephalopathy, EUE); and the like.

In some embodiments, the subject is suspected of having brain injury as a result of having suffered from trauma to the brain selected from the group consisting of traumatic brain injury (TBI), acute infection, acute inflammation, encephalitis, meningitis, non-communicating hydrocephalus, normal pressure hydrocephalus, chronic traumatic encephalopathy (CTE), chronic traumatic encephalomyopathy (CTEM), dementia pugilistica, a symptomatic concussion, and an asymptomatic sub-concussive incident. In some embodiments, the trauma comprises CTEM.

In some embodiments, the method can be used to distinguish the type of brain injury from other types of brain injury. For example, some embodiments may provide differentiation of the brain injury from brain diseases and dementias, including chronic protein misfolding disorders (PMDs). Chronic PMDs may include: amyloidosis such as Alzheimer's disease (AD) or systemic amyloidosis; synucleinopathies such as Parkinson's disease (PD), Lewy body dementia; multiple system atrophy; and synuclein-related neuroaxonal dystrophy; type 2 diabetes; triplet repeat disorders such as Huntington's disease (HD); amyotrophic lateral sclerosis (ALS); and the like.

In further embodiments, the method may include distinguishing the brain injury from a chronic neurodegenerative disease that is primarily associated with biochemical damage. For example, the method may include distinguishing the brain injury from a chronic protein misfolding disorder (PMD) according to the presence or amount of the misfolded tau protein in the biological sample. The chronic PMD may include at least one of: an amyloidosis; a synucleinopathy; a triplet repeat disorder; amyotrophic lateral sclerosis; Alzheimer's disease; systemic amyloidosis; Parkinson's disease; Lewy body dementia; multiple system atrophy; synuclein-related neuroaxonal dystrophy; and Huntington's disease.

In some embodiments, the method may further include determining or evaluating the presence of a brain injury in the subject according to the presence of the misfolded tau protein in the biological sample. In other embodiments, the method includes determining or evaluating the presence of a brain injury by comparing a biological sample obtained from the subject to a control biological sample taken from a control subject. The method may include determining or diagnosing the presence of a brain injury in the subject according to an amount of the misfolded tau protein in the biological sample compared to a predetermined threshold amount. The method may include determining or diagnosing the presence of a brain injury in the subject according to the presence of the misfolded tau protein in the biological sample when the subject exhibits no clinical signs of brain injury according to cognitive testing. The method may include determining or diagnosing the presence of a brain injury in the subject as a contributing factor to one or more clinical signs of brain injury in the subject according to the presence of the misfolded tau protein in the biological sample, the subject exhibiting one or more clinical signs of brain injury according to cognitive testing. The method may include determining or diagnosing the presence of a brain injury in the subject according to the presence of the misfolded tau protein in the biological sample, the subject exhibiting no clinical signs of brain injury according to cognitive testing. The method may include determining or diagnosing the presence of a brain injury in the subject according to a progression or homeostasis of a brain injury in the subject by comparing the amount of the misfolded tau protein in the biological sample to an amount of the misfolded tau protein in a comparison biological sample taken from the subject at a different time compared to the biological sample.

As used herein, “tau proteins” are proteins that are the product of alternative splicing from a single gene that is designated MAPT (microtubule-associated protein tau) in humans. Tau proteins include up to full-length and truncated forms of any of tau's isoforms. Various isoforms include, but are not limited to, the six tau isoforms known to exist in human brain tissue, which correspond to alternative splicing in exons 2, 3, and 10 of the tau gene. Three isoforms have three binding domains and the other three have four binding domains. Misfolded tau may be present in brains of individuals suffering from AD or suspected of having AD, or other tauopathies that, like AD, regard misfolding in the presence of both 4R and 3R tau isoforms. Misfolded tau may also be present in diseases that regard misfolding of primarily 4R tau isoforms, such as progressive supranuclear palsy (PSP), tau-dependent frontotemporal dementia (FTD), corticobasal degeneration (CBD), mild cognitive impairment (MCI), argyrophilic grain disease (AgD), and the like. For additional description of Tau protein isoforms, see Buée et al., Brain Res Brain Res Rev. 33(1), p. 95-130 (2000). Alternative splicing of exon 10 in the tau primary transcript gives rise to protein isoforms with three (3R) or four (4R) microtubule binding repeats, referred to herein as the 3R and 4R tau proteins, respectively. In some embodiments, the tau protein comprises a 3R tau protein, while in further embodiments the tau protein comprises a 4R tau protein.

The present invention describes the use of tau proteins to diagnose and characterize brain injury and neurodegenerative diseases. However, amplification and detection of other misfolding proteins can also be used to characterize brain injury and neurodegenerative diseases. These include prions, which are known to cause TSE, beta amyloid protein, which is involved in Alzheimer's disease, α-synuclein, which is involved in Parkinson's disease, and TDP-43, which is involved in age-related TDP-43 encephalopathy (LATE). See Nelson et al., “Limbic-predominant age-related TDP-43 encephalopathy (LATE): consensus working group,” Brain, Apr. 30, 2019. These misfolded proteins exhibit significantly different characteristics than tau protein, but can provide useful information regarding brain injury. The detection of the presence and/or amount of these other misfolded proteins can be used in particular to better characterize the nature of the brain injury.

As used herein, “Aβ” or “beta amyloid” refers to a peptide formed via sequential cleavage of the amyloid precursor protein (APP). Various Aβ isoforms may include 38-43 amino acid residues. The Aβ protein may be formed when APP is processed by β- and/or γ-secretases in any combination. The Aβ may be a constituent of amyloid plaques in brains of individuals suffering from or suspected of having Alzheimer's disease (AD). Various Aβ isoforms may include and are not limited to Abeta40 and Abeta42. Various Aβ peptides may be associated with neuronal damage associated with AD.

As used herein, “αS” or “alpha-synuclein” refers to full-length, 140 amino acid α-synuclein protein, e.g., “αS-140.” Other isoforms or fragments may include “αS-126,” alpha-synuclein-126, which lacks residues 41-54, e.g., due to loss of exon 3; and “αS-112” alpha-synuclein-112, which lacks residue 103-130, e.g., due to loss of exon 5. The αS may be present in brains of individuals suffering from Parkinson's Disease (PD) or suspected of having PD. Various αS isoforms may include and are not limited to αS-140, αS-126, and αS-112. Various αS peptides may be associated with neuronal damage associated with PD.

In some embodiments, the method includes carrying out at least two or more amplification reactions; and characterizing the risk of brain injury in the subject by comparing, between each of the at least two or more amplification reactions, the presence or amount of misfolded tau protein in each biological sample. For example, the misfolded tau protein can be distinguished using the at least two or more amplification reactions according to one or more features comprising: an amino acid sequence, a post translational modification (PTM), an isoform, a misfolding conformation variant, an aggregation variant; and an amplification kinetics parameter; and characterizing the risk of brain injury in the subject comprises comparing, between each of the at least two or more amplification reactions, the presence or amount of each distinguished misfolded tau protein in each biological sample.

The present method involves determining the level of misfolded tau protein in one or more biological samples. Biological samples include amniotic fluid; bile; plasma; blood; cerebrospinal fluid; cerumen; skin; exudate; feces; gastric fluid; lymph; milk; mucus; mucosal membrane; nasal secretions; peritoneal fluid; plasma; pleural fluid; pus; saliva; sebum; semen; sweat; synovial fluid; tears; urine; and a tissue, e.g., a homogenized or liquefied tissue. In some embodiments, the biological sample is a cerebrospinal fluid sample. Where multiple amplification reactions are being used, a plurality of biological samples, from the same or different sources, can be provided or obtained. In some embodiments, the method includes providing a separate biological sample for each of the one or more amplification reactions.

In some embodiments, the biological sample being a pre-mortem biological sample. A pre-mortem biological sample is one that was obtained from the subject while the subject was alive. Alternately, the biological sample can be a post-mortem biological sample, obtained from a subject that has died.

In some embodiments, the biological sample is obtained from the subject within a certain period of time from the occurrence of a brain trauma. The method may include drawing at least one biological sample from the subject within a time period from the brain trauma of 14 days, 7 days, 3 days, 48 hours, 36 hours, 24 hours, 12 hours, 6 hours, 4 hours, 3 hours, 2 hours, and 1 hour.

The method can include the step of obtaining a biological sample from the subject, or the method can use a provided sample that was obtained earlier. A biological sample may be fresh or stored (e.g. blood or blood fraction stored in a blood bank). Samples can be stored for varying amounts of time, such as being stored for an hour, a day, a week, a month, or more than a month. The biological sample may be a bodily fluid expressly obtained for the assays of this invention or a bodily fluid obtained for another purpose which can be sub-sampled for the assays of this invention.

In some embodiments, the method further comprises selectively concentrating the misfolded tau protein in one or more of the biological sample and the incubation mixture. Selectively concentrating the misfolded tau protein comprises one or more of: pre-treating the biological sample prior to forming the incubation mixture; pre-treating the incubation mixture prior to incubating the incubation mixture; and contacting one or more misfolded tau protein specific antibodies to the misfolded tau protein to form a captured misfolded tau protein, the one or more misfolded tau protein-specific antibodies comprising one or more of: an antibody specific for an amino acid sequence of the misfolded tau protein and an antibody specific for a conformation of the misfolded tau protein. Antibodies specific for the amino acid sequence or conformation of misfolded tau protein can be detected using one or more of: a Western Blot assay, a dot blot assay, and an ELISA. An antibody “specifically binds” when the antibody preferentially binds a target structure, or subunit thereof, but binds to a substantially lesser degree or does not bind to a biological molecule that is not a target structure.

The one or more one or more antibodies capable of binding the misfolded tau protein may be coupled to a solid phase. The solid phase may include one or more of a magnetic bead and a multi-well plate. For example, ELISA plates may be coated with the antibodies used to capture misfolded tau protein from the biological sample. The antibody-coated ELISA plates may be incubated with a biological sample, unbound materials may be washed off, and the PMCA reaction may be performed. Antibodies may also be coupled to beads. The beads may be incubated with the biological sample and used to separate misfolded tau protein-antibody complexes from the remainder of the biological sample.

In some embodiments, the method may include conducting the one or more amplification reactions by conducting one of protein misfolding cyclic amplification (PMCA) and quaking-induced conversion (QuIC). Such techniques have been shown to provide ultra-sensitive detection of misfolded aggregates through artificial acceleration and amplification of the misfolding and aggregation process in vitro. The basic concept of PMCA is known, including specific conditions for various proteins (e.g., Soto et al, WO/2002/004954; Estrada, et al. U.S. Pat. App. Pub. No. 20080118938; Soto et al. U.S. Pat. No. 9,910,049; Soto et al. U.S. patent application Ser. Nos. 14/852,475, 14/852,478, Soto et al, U.S. App. No. TBD, filed May 16, 2018, Atty. Docket. No. Amprion-SOUS, and claiming priority to U.S. Provisional Pat. No. 62/507,166; and corresponding the; Saijo, et al., Acta Neuropathol 2017, 133:75 1-765); each of which is entirely incorporated herein by reference). The basic concept of QuiC is known, including specific conditions for various proteins (e.g., Atarashi, et al. Prion. 2011 July-September; 5(3): 150-153; Schmitz, et al., Nat Protoc. 2016 November; 11(11):2233-2242; each of which is entirely incorporated herein by reference). For a description of amplification methods as specifically applied to Tau proteins, see U.S. patent application Ser. No. 15/981,449, “Detection of Misfolded Tau Protein,” the disclosure of which is incorporated herein by reference.

In various embodiments, methods for determining a presence of a misfolded tau protein in a sample are provided. The methods may include capturing misfolded tau protein from the sample. The methods may include contacting the captured misfolded tau protein with a molar excess of monomeric, folded tau protein to form an incubation mixture. The molar excess may be greater than an amount of protein monomer included in the captured misfolded tau protein. The methods may include conducting an incubation cycle two or more times effective to form an amplified portion of misfolded tau protein. Each incubation cycle may include incubating the incubation mixture effective to cause misfolding and/or aggregation of at least a portion of the monomeric, folded tau protein in the presence of the captured misfolded tau protein to form an amplified portion of misfolded tau protein. Each incubation cycle may include physically disrupting the incubation mixture effective to break up at least a portion of any protein aggregate present, e.g., to release the misfolded tau protein. The methods may also include determining the presence of the misfolded tau protein in the sample by detecting at least a portion of the misfolded tau protein. The misfolded tau protein may include one or more of: a misfolded monomer and a misfolded aggregate. The captured misfolded tau protein may include one or more of: a captured misfolded monomer and a captured misfolded aggregate. The amplified portion of misfolded tau protein may include one or more of: an amplified portion of the misfolded monomer, an amplified portion of the misfolded aggregate, and insoluble misfolded aggregate.

As used herein, references to the misfolded tau protein may include any form of the misfolded tau protein, distributed in the sample, the incubation mixture, and the like. For example, references to the misfolded tau protein may include the misfolded tau protein, for example, the misfolded tau protein in a sample from a subject suffering from a brain injury. References to the misfolded tau protein may include, for example, the amplified portion of misfolded tau protein, e.g., in the incubation mixture. References to the misfolded tau protein may include the captured misfolded tau protein, e.g., misfolded tau protein captured from the sample using misfolded tau protein-specific antibodies.

In some embodiments, the methods may include contacting an indicator of the misfolded tau protein to the incubation mixture. The indicator of the misfolded tau protein may be characterized by an indicating state in the presence of the misfolded tau protein and a non-indicating state in the absence of the misfolded tau protein. The determining the presence of the misfolded tau protein in the sample may include detecting the indicating state of the indicator of the misfolded tau protein. The indicating state of the indicator and the non-indicating state of the indicator may be characterized by a difference in fluorescence. The determining the presence of the misfolded tau protein in the sample may include detecting the difference in fluorescence.

In several embodiments, the method may include contacting a molar excess of the indicator of the misfolded tau protein to the incubation mixture. The molar excess may be greater than a total molar amount of protein monomer included in the monomeric, folded tau protein and the misfolded tau protein in the incubation mixture.

In various embodiments, the indicator of the misfolded tau protein may include one or more of: Thioflavin T, Congo Red, m-I-Stilbene, Chrysamine G, PIB, BF-227, X-34, TZDM, FDDNP, MeO-X-04, IMPY, NIAD-4, luminescent conjugated polythiophenes, a fusion with a fluorescent protein such as green fluorescent protein and yellow fluorescent protein, derivatives thereof, and the like.

In various embodiments, methods for determining a presence of a misfolded tau protein in a sample are provided. The methods may include contacting the sample with Thioflavin T and a molar excess of a monomeric, folded tau protein to form an incubation mixture. The molar excess may be greater than an amount of protein monomer included in the misfolded tau protein in the sample. The methods may include conducting an incubation cycle two or more times effective to form an amplified portion of misfolded tau protein. Each incubation cycle may include incubating the incubation mixture effective to cause misfolding and/or aggregation of at least a portion of the monomeric, folded tau protein in the presence of the misfolded tau protein to form the amplified portion of misfolded tau protein. Each incubation cycle may include shaking the incubation mixture effective to break up at least a portion of any protein aggregate present, e.g., to release the misfolded tau protein. The methods may also include determining the presence of the misfolded tau protein in the sample by detecting a fluorescence of the Thioflavin T corresponding to misfolded tau protein. The misfolded tau protein may include one or more of: a misfolded monomer and a misfolded aggregate. The captured misfolded tau protein may include one or more of: a captured misfolded monomer and a captured misfolded aggregate. The amplified portion of misfolded tau protein may include one or more of: an amplified portion of the misfolded monomer, an amplified portion of the misfolded aggregate, and a misfolded aggregate.

In various embodiments, the contacting the sample with the monomeric, folded tau protein to form the incubation mixture may include contacting a molar excess of the monomeric, folded tau protein to the sample including the captured misfolded tau protein. The molar excess of the monomeric, folded tau protein may be greater than a total molar amount of protein monomer included in the captured misfolded tau protein. The incubating the incubation mixture may be effective to cause misfolding and/or aggregation of at least a portion of the monomeric, folded tau protein in the presence of the captured misfolded tau protein to form the amplified portion of misfolded tau protein.

In some embodiments, the protein aggregate may include one or more of: the monomeric protein, the misfolded tau protein, and a captured form of the misfolded tau protein.

In several embodiments, the physically disrupting the incubation mixture may include one or more of: sonication, stirring, shaking, freezing/thawing, laser irradiation, autoclave incubation, high pressure, homogenization, and the like. For example, shaking may include cyclic agitation. The cyclic agitation may be conducted between about 50 rotations per minute (RPM) and 10,000 RPM. The cyclic agitation may be conducted between about 200 RPM and about 2000 RPM. The cyclic agitation may be conducted at about 500 RPM.

In various embodiments, the physically disrupting the incubation mixture may be conducted in each incubation cycle for between about 5 seconds and about 10 minutes, between about 30 sec and about 1 minute, between about 45 sec and about 1 minute, for about 1 minute, and the like. For example, the physically disrupting the incubation mixture may be conducted in each incubation cycle by shaking for one or more of: between about 5 seconds and about 10 minutes, between about 30 sec and about 1 minute, between about 45 sec and about 1 minute, for about 1 minute, and the like. The incubating the incubation mixture may be independently conducted, in each incubation cycle, for a time between about 5 minutes and about 5 hours, between about 10 minutes and about 2 hours, between about 15 minutes and about 1 hour, between about 25 minutes and about 45 minutes, and the like. Each incubation cycle may include independently incubating and physically disrupting the incubation mixture for one or more of: incubating between about 5 minutes and about 5 hours and physically disrupting between about 5 seconds and about 10 minutes; incubating between about 10 minutes and about 2 hours and physically disrupting between about 30 sec and about 1 minute; incubating between about 15 minutes and about 1 hour and physically disrupting between about 45 sec and about 1 minute; incubating between about 25 minutes and about 45 minutes and physically disrupting between about 45 sec and about 1 minute; and incubating about 1 minute and physically disrupting about 1 minute.

The conducting the incubation cycle may be repeated between about 2 times and about 1000 times, between about 5 times and about 500 times, between about 50 times and about 500 times, between about 150 times and about 250 times, and the like. The incubating the incubation mixture being independently conducted, in each incubation cycle, at a temperature in ° C. of about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or a range between any two of the preceding values, for example, between about 15° C. and about 50° C.

In several embodiments, contacting the sample with the monomeric, folded tau protein to form the incubation mixture may be conducted under physiological conditions. Contacting the sample with the monomeric, folded tau protein to form the incubation mixture may include contacting the sample with a molar excess of the monomeric protein. The molar excess may be greater than a total molar amount of protein monomer included in the misfolded tau protein in the sample. The monomeric, folded tau protein and/or the misfolded tau protein may include one or more peptides, e.g., formed by proteolytic cleavage of the monomeric, folded tau protein and/or the misfolded tau protein.

In various embodiments, the monomeric, folded tau protein may be produced by one of: chemical synthesis, recombinant production, or extraction from non-recombinant biological samples. The misfolded tau protein may substantially be the misfolded aggregate. The amplified portion of misfolded tau protein substantially being one or more of: the amplified portion of the misfolded aggregate and the misfolded aggregate.

In various embodiments, conducting the one or more amplification reactions may include conducting at least two or more amplification reactions. The method may include evaluating the brain injury in the subject by comparing, between each of the at least two or more amplification reactions, the presence or amount of each misfolded tau protein in each biological sample.

In some embodiments, conducting the one or more amplification reactions may include conducting at least two or more amplification reactions. The misfolded tau proteins may be distinguished between the at least two or more amplification reactions according to one or more features. The one or more features may include an amino acid sequence, a post translational modification (PTM), an isoform, a misfolding conformation variant, an aggregation variant; and an amplification kinetics parameter. The method may include characterizing the brain injury in the subject by comparing, between each of the at least two or more amplification reactions, the presence or amount of each distinguished misfolded tau protein in each biological sample.

In various embodiments, conducting the one or more amplification reactions may include conducting at least two or more amplification reactions in which each biological sample is drawn from a distinct reservoir in the subject. The distinct reservoir may include a source in the body of each biological fluid, for example, the cerebrospinal system and the vasculature may provide distinct reservoirs for cerebrospinal fluid and blood, respectively. Evaluating the brain injury in the subject may include comparing, between each of the at least two or more amplification reactions, the presence or amount of each distinct misfolded tau protein in each biological sample from each distinct reservoir in the subject.

In some embodiments, conducting the one or more amplification reactions may include conducting at least two or more amplification reactions in which each biological sample is drawn from the subject at a distinct sampling time. Evaluating the brain injury in the subject may include comparing, between each of the at least two or more amplification reactions, the presence or amount of each distinct misfolded tau protein in each biological sample at each distinct sampling time. The method may include drawing at least one biological sample from the subject within a time period from the brain injury or suspected brain injury of 14 days, 7 days, 3 days, 48 hours, 36 hours, 24 hours, 12 hours, 6 hours, 4 hours, 3 hours, 2 hours, and 1 hour.

In several embodiments, the detecting the misfolded tau protein may include one or more of: a Western Blot assay, a dot blot assay, an enzyme-linked immunosorbent assay (ELISA), a thioflavin T binding assay, a Congo Red binding assay, a sedimentation assay, electron microscopy, atomic force microscopy, surface plasmon resonance, and spectroscopy. The ELISA may include a two-sided sandwich ELISA. The spectroscopy may include one or more of: quasi-light scattering spectroscopy, multispectral ultraviolet spectroscopy, confocal dual-color fluorescence correlation spectroscopy, Fourier-transform infrared spectroscopy, capillary electrophoresis with spectroscopic detection, electron spin resonance spectroscopy, nuclear magnetic resonance spectroscopy, Fluorescence Resonance Energy Transfer (FRET) spectroscopy, and the like.

In various embodiments, the detecting the misfolded tau protein may include contacting the incubation mixture with a protease. The misfolded tau protein may be detected using anti-misfolded tau protein antibodies in one or more of: a Western Blot assay, a dot blot assay, and an ELISA.

In some embodiments, the method may include providing the monomeric, folded tau protein in labeled form. The monomeric, folded tau protein in labeled form may include one or more of: a covalently incorporated radioactive amino acid, a covalently incorporated, isotopically labeled amino acid, and a covalently incorporated fluorophore. The detecting the misfolded tau protein include detecting the monomeric, folded tau protein in labeled form as incorporated into the amplified portion of misfolded tau protein.

In some embodiments, determining the presence of the misfolded tau protein in the sample may include determining an amount of the misfolded tau protein in the sample. The amount of the misfolded tau protein in the sample may be determined compared to a control sample. The amount of the misfolded tau protein in the sample may be detected with a sensitivity of at least about one or more of: 80%, 81%, 82%, 83%, 84%, 85%, 86%, 87%, 88%, 89%, 90%, 91%, 92%, 93%, 94%, 95%, 96%, 97%, 98%, 99%, and 100%. The amount of the misfolded tau protein in the sample detected may be less than about one or more of: 100 nmol, 10 nmol, 1 nmol, 100 pmol, 10 pmol, 1 pmol, 100 fmol, 10 fmol, 3 fmol, 1 fmol, 100 attomol, 10 attomol, and 1 attomol. The amount of the misfolded tau protein in the sample may be detected in a molar ratio to monomeric, folded tau protein comprised by the sample. The molar ratio may be less than about one or more of 1:100, 1:10,000, 1:100,000, and 1:1,000,000.

In various embodiments, the misfolded tau protein in the sample may be detected with a specificity of at least about one or more of: 80%, 81%, 82%, 83%, 84%, 85%, 86%, 87%, 88%, 89%, 90%, 91%, 92%, 93%, 94%, 95%, 96%, 97%, 98%, 99%, and 100%.

In some embodiments, the incubation mixture may include the monomeric, folded tau protein in a concentration, or in a concentration range, of one or more of: between about 1 nM and about 2 mM; between about 10 nM and about 200 μM; between about 100 nM and about 20 μM; or between about 1 μM and about 10 μM; and about 2 μM.

In several embodiments, the incubation mixture may include a buffer composition. The buffer composition may be effective to prepare or maintain the pH of the incubation mixture as described herein, e.g., between pH 5 and pH 9. The buffer composition may include one or more of: Tris-HCL, PBS, MES, PIPES, MOPS, BES, TES, and HEPES, and the like. The buffer concentration may be at a total concentration of between about 1 μm and about 1M. For example, the buffer may be Tris-HCL at a concentration of 0.1 M.

In various embodiments, the incubation mixture may include a salt composition. The salt composition may be effective to increase the ionic strength of the incubation mixture. The salt composition may include one or more of: NaCl, KCl, and the like. The incubation mixture may include the salt composition at a total concentration of between about 1 μm and about 500 mM.

In several embodiments, the incubation mixture may be characterized by, prepared with, or maintained at a pH value of or a pH range of one or more of: between about 5 and about 9; between about 6 and about 8.5; between about 7 and about 8; and about 7.4.

In some embodiments, the incubation mixture may be incubated at a temperature in ° C. of about one or more of: 4, 6, 8, 10, 12, 14, 16, 18, 20, 22, 24, 26, 28, 30, 32, 34, 35, 36, 37, 40, 45, 50, 55, and 60, e.g., about 22° C., or a temperature range between any two of the preceding values, for example, one or more of: between about 4° C. and about 60° C.; between about 4° C. and about 35° C.; between about 8° C. and about 50° C.; between about 12° C. and about 40° C.; between about 18° C. and about 30° C.; between about 18° C. and about 26° C.; and the like.

In some embodiments, evaluating the brain injury in the subject according to the presence or amount of each misfolded tau protein in each biological sample may include classifying the misfolded tau protein according to one or more features. The one or more features may include: an amino acid sequence, a post translational modification (PTM) such as phosphorylation or glycosylation, an isoform, a misfolding conformation variant, an aggregation variant and an amplification kinetics parameter. The method may include determining the one or more features using one or more of: protein sequencing; an antibody; an indicator; chemical analysis of the PTM; a spectrum; microscopy, a proteolytic resistance; a stability to denaturation; and a kinetics analysis of the one or more amplification reactions. For example, certain misfolding conformation variants may be distinguished by the aggregates they form, such as fibrils.

In some embodiments, a subject identified as having an increased risk of brain injury is provided with treatment for brain injury. The nature of the treatment of the brain injury will depend on the specific type of brain injury present in the subject, with significant differences existing between treatment of acute and chronic brain injury.

The treatment of acute brain injury focuses on assuring the person has enough oxygen from the brain blood supply, and on maintaining normal blood pressure to avoid further injuries of the head or neck. The person may need surgery to remove clotted blood or repair skull fractures. Medicines used for traumatic injuries are diuretics, anti-seizure or coma-inducing drugs. Diuretics reduce the fluid in tissues lowering the pressure on the brain. Coma-inducing drugs may be used during surgery to reduce impairments and restore blood flow.

The treatment of chronic brain injury often involves rehabilitation assisted by experts specializing in the treatment of brain injury. For example, occupational therapists may be involved in running rehabilitation programs to help restore lost function or help re-learn essential skills. Other treatments for chronic brain injury include medication, psychotherapy, neuropsychological rehabilitation, snoezelen, surgery, or physical implants such as deep brain stimulation.

A further example of chronic brain injury is a protein misfolding disorder. Treatments are known to those skilled in the art for various different protein misfolding disorders. For example, for Alzheimer's Disease (AD), the treatment may include administration of one or more of: an inhibitor of BACE1 (beta-secretase 1); an inhibitor of γ-secretase; and a modulator of Aβ homeostasis, e.g., an immunotherapeutic modulator of Aβ homeostasis. The Aβ modulating therapy may include administration of one or more of: E2609; MK-8931; LY2886721; AZD3293; semagacestat (LY-450139); avagacestat (BMS-708163); solanezumab; crenezumab; bapineuzumab; BIIB037; CAD106; 8F5 or 5598 or other antibodies raised against Aβ globulomers, e.g., as described by Barghorn et al, J. Neurochem., 2005, 95, 834-847, the entire teachings of which are incorporated herein by reference; ACC-001; V950; Affitrope AD02; and the like.

For Parkinson's Disease (PD), the treatment may include active immunization, such as PD01A+ or PDO3A+, passive immunization such as PRX002, and the like. The PMD modulating therapy may also include treatment with GDNF (Glia cell-line derived neurotrophic factor), inosine, Calcium-channel blockers, specifically Cav1.3 channel blockers such as isradipine, nicotine and nicotine-receptor agonists, GM-CSF, glutathione, PPAR-gamma agonists such as pioglitazone, and dopamine receptor agonists, including D2/D3 dopamine receptor agonists and LRRK2 (leucine-rich repeat kinase 2) inhibitors.

In some embodiments, methods including a treatment step further comprise comparing the amount of the misfolded tau protein in the biological sample to an amount of the misfolded tau protein in a comparison biological sample, the biological sample and the comparison biological sample being taken from the subject at different times over a period of time under the treatment; and determining if the subject is responsive to the treatment according to a change in the misfolded tau protein over the period of time, or non-responsive to the treatment according to homeostasis of the misfolded tau protein over the period of time.

### Evaluating the Risk of Neurodegenerative Disease or Disorder

Another aspect of the invention provides a method for evaluating the risk of neurodegenerative disease or disorder in a subject having suffered from brain trauma. The method includes providing one or more biological sample from the subject; conducting one or more amplification reactions, each amplification reaction comprising: contacting a portion of the biological sample with a monomeric, folded tau protein to form an incubation mixture; subjecting the incubation mixture to amplification conditions that are effective, in the presence of the misfolded tau protein, to form an amplified portion of the misfolded tau protein from the monomeric, folded tau protein; detecting the amplified portion of the misfolded tau protein; determining a presence or amount of the misfolded tau protein in the biological sample according to the amplified portion of the misfolded tau protein; and characterizing the risk of neurodegenerative disease in the subject according to the presence or amount of the misfolded tau protein in the biological sample.

Without wishing to be bound by theory, it is believed that some neurodegenerative diseases or disorders may result from brain trauma. For example, brain trauma associated with concussive behaviors in horned or antlered animals can lead to neurodegenerative diseases or disorders such as transmissible spongiform encephalitis (TSE) which can later be transmitted by infectious means. In some embodiments, the brain trauma is traumatic brain injury, while in further embodiments the traumatic brain injury is chronic traumatic encephalopathy.

Neurodegeneration is the progressive loss of structure or function of neurons, including death of neurons. Many neurodegenerative diseases or disorders—including amyotrophic lateral sclerosis, Parkinson's disease, Alzheimer's disease, and Huntington's disease—occur as a result of neurodegenerative processes. Some neurodegenerative diseases are caused by genetic mutations, protein degradation, or mitochondrial dysfunction. In some embodiments, neurological disease or disorder is a chronic protein misfolding disorder (PMD) selected from the group consisting of an amyloidosis; a synucleinopathy; a triplet repeat disorder; amyotrophic lateral sclerosis; Alzheimer's disease; systemic amyloidosis; Parkinson's disease; Lewy body dementia; multiple system atrophy; synuclein-related neuroaxonal dystrophy; and Huntington's disease. In some embodiments, the method of the invention can include selecting a subject that is exhibiting the one or more clinical signs of dementia according to cognitive testing.

In some embodiments, the neurological disease or disorder is a is a tauopathy selected from the group consisting of Alzheimer's disease (AD), Parkinson's Disease (PD), Progressive Supranuclear Palsy (PSP), FrontoTemporal Dementia (FTD), Corticobasal degeneration (CBD), Mild cognitive impairment (MCI), Argyrophilic grain disease (AgD) Traumatic Brain Injury (TBI), Chronic Traumatic Encephalopathy (CTE), and Dementia Pugilistica (DP).

In some embodiments, the tauopathy is a primary tauopathy or a secondary tauopathy. The tauopathy may be characterized at least in part by misfolding and/or aggregation of 4R tau protein. The tauopathy may be characterized at least in part by misfolding and/or aggregation of 4R tau protein and 3R tau protein. The tauopathy may be characterized at least in part by misfolded and/or aggregated 4R tau protein, in a ratio to misfolded and/or aggregated 3R tau protein, of one of about: 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, and 99:1, or a range between any two of the preceding ratios, for example, between 1:99 and 99:1.

In several embodiments, the methods may include characterizing an identity of the tauopathy by analyzing the first amplified, misfolded protein aggregate or one or more corresponding PMCA kinetic parameters thereof for a signature of at least one of: Alzheimer's disease (AD), Parkinson's Disease (PD), Progressive Supranuclear Palsy (PSP), FrontoTemporal Dementia (FTD), Corticobasal degeneration (CBD), Mild cognitive impairment (MCI), Argyrophilic grain disease (AgD) Traumatic Brain Injury (TBI), Chronic Traumatic Encephalopathy (CTE), and Dementia Pugilistica (DP). For example, characterizing the identity of the tauopathy may include determining the one or more corresponding PMCA kinetic parameters, including one or more of: lag phase, T₅₀, amplification rate, and amplification extent. Characterizing the identity of the tauopathy may include comparing the one or more corresponding PMCA kinetic parameters to one or more corresponding predetermined corresponding PMCA kinetic parameters that are characteristic of the identity of the tauopathy to determine a similarity or difference effective to characterize the identity of the tauopathy.

In some embodiments, the methods may include characterizing the identity of the tauopathy using an antibody selective for a conformational epitope of a tauopathy-specific misfolded tau protein aggregate. The methods may include characterizing the identity of the tauopathy using an indicator selective for each tauopathy-specific misfolded tau protein aggregate. The indicator selective for each tauopathy-specific misfolded tau protein aggregate may include a small molecule, a peptide, or a DNA or RNA aptamer; and the like. The methods may include characterizing the identity of the tauopathy using a spectrum characteristic of each tauopathy-specific misfolded tau protein aggregate.

In some embodiments, the methods may include, for example, characterizing the identity of the tauopathy by analyzing the proteolytic resistance of each tauopathy-specific misfolded tau protein aggregate. For example, each tauopathy-specific misfolded tau protein aggregate may be contacted with a proteinase, e.g., proteinase K, trypsin, chymotrypsin, and the like, at a proteinase concentration of from 0.1 to 5000 μg/mL, at various temperatures from 20° C. to 120° C. and for various times, e.g., from 1 min to 4 h. The proteolytic resistance of each tauopathy-specific misfolded tau protein aggregate may be characterized and used to distinguish the various tauopathy-specific misfolded tau protein aggregates.

In several embodiments, the methods may include characterizing the identity of the tauopathy by analyzing the stability to denaturation of each tauopathy-specific misfolded tau protein aggregate. For example, each tauopathy-specific misfolded tau protein aggregate may be treated with guanidinium or urea at a sufficiently elevated temperature to induce protein denaturation of each tauopathy-specific misfolded tau protein aggregate. The concentration of guanidinium or urea may range from 0.1 M to 8 M. The temperature may range between 20° C. to 120° C. The stability of each tauopathy-specific misfolded tau protein aggregate may be characterized and used to distinguish the various tauopathy-specific misfolded tau protein aggregates.

The methods may include sedimentation of each tauopathy-specific misfolded tau protein aggregate. The methods may include gel chromatography to characterize the size of each tauopathy-specific misfolded tau protein aggregate. The methods may include circular dichroism spectroscopy of each tauopathy-specific misfolded tau protein aggregate. The methods may include Fourier transform infrared spectroscopy to analyze secondary structure of each tauopathy-specific misfolded tau protein aggregate. The methods may include nuclear magnetic resonance spectroscopy to analyze structure of each tauopathy-specific misfolded tau protein aggregate. The methods may include mass spectrometry, e.g., fragmentation and collision induced dissociation to analyze secondary and tertiary structure of each tauopathy-specific misfolded tau protein aggregate. The methods may include microscopy, e.g., atomic force microscopy, cryo-electron microscopy, and the like to analyze morphology of each tauopathy-specific misfolded tau protein aggregate. Each of these methods may be coupled with substitution using atomic isotopes of different mass, magnetic properties, and/or isotopic stability to complement the methods; for example, nuclear magnetic resonance spectroscopy may be coupled with deuterium exchange in each tauopathy-specific misfolded tau protein aggregate to obtain structural information.

In various embodiments, the methods are provided such that the tauopathy specifically excludes Pick's disease. In various embodiments, the exclusion of Pick's disease does not encompass the remainder of Pick's complex of diseases.

In several embodiments, the methods may include determining or diagnosing the presence or absence of a tauopathy in the subject including comparing the presence or absence of the first misfolded protein aggregate in the sample to a control sample taken from a control subject. The detecting may include detecting an amount of the first misfolded protein aggregate in the sample. The sample may be taken from a subject. The methods may include determining or diagnosing the presence or absence of a tauopathy in the subject by comparing the amount of the first misfolded protein aggregate in the sample to a predetermined threshold amount. The sample may be taken from a subject exhibiting no clinical signs of dementia according to cognitive testing. The methods may include determining or diagnosing the presence or absence of a tauopathy in the subject according to the presence or absence of the first misfolded protein aggregate in the sample. The sample may be taken from a subject exhibiting no cortex plaques or tangles according to contrast imaging. The methods may include determining or diagnosing the presence or absence of a tauopathy in the subject according to the presence or absence of the first misfolded protein aggregate in the sample. The sample may be taken from a subject exhibiting clinical signs of dementia according to cognitive testing. The methods may include determining or diagnosing the presence or absence of a tauopathy as a contributing factor to the clinical signs of dementia in the subject according to the presence or absence of the first misfolded protein aggregate in the sample. The sample may be taken from a subject exhibiting no clinical signs of dementia according to cognitive testing. The subject may exhibit a predisposition to dementia according to genetic testing. The genetic testing may indicate, for example, an increased risk of tauopathy according to one or two copies of the ApoE4 allele, variants of the brain derived neurotrophic factor (BDNF) gene, such as the va166met allele, in which valine at AA position 66 is replaced by methionine, and the like. The methods may include determining or diagnosing the presence or absence of a tauopathy in the subject according to the presence or absence of the first misfolded protein aggregate in the sample.

### Kits

In another aspect of the invention, a kit is provided for determining a presence or amount in a sample of a misfolded tau protein. The presence or amount of the misfolded tau protein or misfolded tau protein aggregate can be used to evaluate the likelihood that the subject has suffered a brain injury, or the risk that a subject having a brain injury will develop a neurological disease or disorder such as a tauopathy. The kit may include a tau protein that may include 4R tau. The kit may include an indicator of the misfolded tau protein (e.g., misfolded tau protein aggregate). The misfolded tau protein may correspond to one known to be associated with a tauopathy. The kit may include a buffer. The kit may include heparin. The kit may include a salt. The kit may include instructions. The instructions may direct a user to obtain the sample. The instructions may direct the user to perform at least a PMCA procedure. The PMCA procedure may include forming an incubation mixture by contacting a portion of the sample with the tau protein, the indicator of the misfolded tau protein aggregate, the buffer, the heparin, and the salt. The incubation mixture may be formed with a concentration of one or more of: the tau protein of less than about 20 μM; the heparin of less than about 75 μ; the salt as NaCl of less than about 190 mM; and the indicator of the misfolded tau protein aggregate as Thioflavin T of less than about 9.5 μM. The PMCA procedure may include conducting an incubation cycle two or more times effective to form an amplified, misfolded tau protein. Each incubation cycle may include incubating the incubation mixture effective to cause misfolding and/or aggregation of the tau protein in the presence of the misfolded tau protein (e.g., misfolded tau protein aggregate). Each incubation cycle may include disrupting the incubation mixture effective to form the amplified, misfolded tau protein. The instructions may direct the user to determine the presence or absence in the sample of the misfolded tau protein by analyzing the incubation mixture for the presence or absence of the amplified, misfolded tau protein according to the indicator of the misfolded tau protein.

In several embodiments, the kit may include any element of the methods described herein. Moreover, the kit may include instructions directing the user to conduct any of the steps of the methods described herein.

In some embodiments, for example, the instructions may include directing the user to obtain the sample from a subject. The sample may include one or more of: a bio-fluid, a biomaterial, a homogenized tissue, and a cell lysate. The instructions directing the user to determine or diagnose a tauopathy in the subject according to the presence or absence in the sample of the misfolded tau protein (e.g., misfolded tau protein aggregate).

In some embodiments, the kit may include a PMCA apparatus. The PMCA apparatus may include one or more of: a multiwall microtitre plate; a microfluidic plate; a shaking apparatus; a spectrometer; and an incubator. The apparatus may be included either as one or more of the individual plates or apparatuses, as a combination device, and the like. For example, a shaking microplate reader may be used to perform cycles of incubation and shaking and automatically measure the ThT fluorescence emission during the course of an experiment (e.g., FLUO star OPTIMA, BMG LABTECH Inc., Cary, N.C.).

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method for evaluating a subject for a brain injury, comprising:
providing at least one biological sample from the subject;
conducting one or more amplification reactions, each amplification reaction comprising:
contacting a portion of the biological sample with a monomeric, tau protein to form an incubation mixture;
subjecting the incubation mixture to amplification conditions that are effective, in the presence of the misfolded tau protein, to form an amplified portion of the misfolded tau protein from the monomeric tau protein;
detecting the amplified portion of the misfolded tau protein;
determining the presence or amount of the misfolded tau protein in the biological sample by detecting the presence or amount of the amplified portion of the misfolded tau protein; and
characterizing the subject as having an increased risk of having brain injury if misfolded tau protein is determined to be present in the biological sample.

2. The method of claim 1, wherein the tau protein comprises a 3R tau protein.

3. The method of claim 1, wherein the tau protein comprises a 4R tau protein.

4. The method of claim 1, in which evaluating the risk of brain injury in the subject further comprises classifying the misfolded tau protein according to one or more features comprising: an amino acid sequence, a post translational modification (PTM), an isoform, a misfolding conformation variant, an aggregation variant; and an amplification kinetics parameter.

5. The method of claim 4, wherein the misfolded tau protein is classified using one or more of: protein sequencing; an antibody; an indicator; chemical analysis of the PTM; a spectrum; microscopy, a proteolytic resistance; a stability to denaturation; and a kinetics analysis of the one or more amplification reactions.

6. The method of claim 1, wherein conducting the one or more amplification reactions comprise conducting at least two or more amplification reactions; and
characterizing the risk of brain injury in the subject by comparing, between each of the two or more amplification reactions, the presence or amount of misfolded tau protein in each biological sample.

7. The method of claim 1, wherein conducting the one or more amplification reactions comprises conducting two or more amplification reactions, the misfolded tau protein being distinguished between the two or more amplification reactions according to one or more features comprising: an amino acid sequence, a post translational modification (PTM), an isoform, a misfolding conformation variant, an aggregation variant; and an amplification kinetics parameter; and
characterizing the risk of brain injury in the subject comprises comparing, between each of the two or more amplification reactions, the presence or amount of misfolded tau protein in each biological sample.

8. The method of claim 1, the at least one biological sample being a pre-mortem biological sample.

9. The method of claim 1, further comprising obtaining the at least one biological sample from the subject.

10. The method of claim 1, wherein providing the at least one biological sample from the subject comprises providing a separate biological sample for each of the one or more amplification reactions.

11. The method of claim 1, wherein the biological sample comprises cerebrospinal fluid.

12. The method of claim 1, wherein the one or more amplification reactions comprise protein misfolding cyclic amplification (PMCA) and/or quaking-induced conversion (QuIC).

13. The method of claim 1, wherein the subject is suspected as having brain injury as a result of having suffered from a brain trauma selected from the group consisting of traumatic brain injury (TBI), acute infection, acute inflammation, encephalitis, meningitis, non-communicating hydrocephalus, normal pressure hydrocephalus, chronic traumatic encephalopathy (CTE), chronic traumatic encephalomyopathy (CTEM), dementia pugilistica, a symptomatic concussion, and an asymptomatic sub-concussive incident.

14. The method of claim 13, wherein the brain trauma comprises CTEM.

15. The method of claim 13, wherein the biological sample is obtained from the subject within 24 hours from the occurrence of the brain trauma.

16. The method of claim 1, further comprising selectively concentrating the misfolded tau protein in one or more of the biological sample and the incubation mixture.

17. The method of claim 16, the selectively concentrating the misfolded tau protein comprises one or more of:
pre-treating the biological sample prior to forming the incubation mixture;
pre-treating the incubation mixture prior to incubating the incubation mixture; and contacting one or more misfolded tau protein-specific antibodies to the misfolded tau protein to form a captured misfolded tau protein, the one or more misfolded tau protein specific antibodies comprising one or more of: an antibody specific for an amino acid sequence of the misfolded tau protein and an antibody specific for a conformation of the misfolded tau protein.

18. The method of claim 1, wherein a subject identified as having an increased risk of brain injury is provided with treatment for brain injury.

19. The method of claim 18, wherein the method further comprises comparing the amount of the misfolded tau protein in the biological sample to an amount of the misfolded tau protein in a comparison biological sample, the biological sample and the comparison biological sample being taken from the subject at different times over a period of time under the treatment; and
determining if the subject is responsive to the treatment according to a change in the misfolded tau protein over the period of time, or non-responsive to the treatment according to homeostasis of the misfolded tau protein over the period of time.

20. A method for evaluating the risk of neurodegenerative disease or disorder in a subject having suffered from brain trauma, comprising:
providing one or more biological samples from the subject;
conducting one or more amplification reactions, each amplification reaction comprising:
contacting a portion of the biological sample with a monomeric, folded tau protein to form an incubation mixture;
subjecting the incubation mixture to amplification conditions that are effective, in the presence of the misfolded tau protein, to form an amplified portion of the misfolded tau protein from the monomeric, folded tau protein;
detecting the amplified portion of the misfolded tau protein;
determining a presence or amount of the misfolded tau protein in the biological sample according to the amplified portion of the misfolded tau protein; and
characterizing the risk of neurodegenerative disease in the subject according to the presence or amount of the misfolded tau protein in the biological sample.

21. The method of claim 20, wherein the brain trauma is traumatic brain injury.

22. The method of claim 21, wherein the traumatic brain injury is chronic traumatic encephalopathy.

23. The method of claim 20, wherein the neurological disease or disorder is a is a tauopathy selected from the group consisting of Alzheimer's disease (AD), Parkinson's Disease (PD), Progressive Supranuclear Palsy (PSP), FrontoTemporal Dementia (FTD), Corticobasal degeneration (CBD), Mild cognitive impairment (MCI), Argyrophilic grain disease (AgD) Traumatic Brain Injury (TBI), Chronic Traumatic Encephalopathy (CTE), and Dementia Pugilistica (DP).

24. The method of claim 20, wherein the subject is exhibiting one or more clinical signs of dementia according to cognitive testing.
